(19) United States
(12) Patent Application Publication
(10) Pub. No.: US 2025/0145930 A1
(43) Pub. Date: May 8, 2025

(54) Loading, Distributing, And Expanding Cells In A Hollow Fiber Bioreactor

(71) Applicant: Terumo BCT, Inc., Lakewood, CO (US)

(72) Inventor: Nathan D. FRANK, Lakewood, CO (US)

(21) Appl. No.: 18/929,047

(22) Filed: Oct. 28, 2024

Related U.S. Application Data
(60) Provisional application No. 63/595,366, filed on Nov. 2, 2023.

(51) Int. Cl. C12M 1/26; C12M 1/00

(52) U.S. Cl. CPC C12M 33/04; C12M 23/40; C12M 29/18

(57) ABSTRACT

A method for using a bioreactor-based system includes circulating a volume including a cell population through a space in a first direction for a first period, the space having a first orientation for the first period; circulating the volume in a second direction for a second period, the space having the first orientation for the second period; circulating the volume in the first direction for a third period, the space having a second orientation for the third period; and circulating the volume in the second direction for a fourth period, the space having the second orientation for the fourth period. The method further includes maintaining a culture medium in the space using bidirectional movement at a first rate, causing the culture medium to move through the space at a second rate that is less than the first rate, and causing a washing fluid to move through the space.

### CROSS-REFERENCE TO RELATED APPLICATIONS

This application claims the benefit of U.S. Provisional Application No. 63/595,366 filed on Nov. 2, 2023. The entire disclosure of the above application is incorporated herein by reference.

### FIELD

The present disclosure relates to systems and methods for producing, or preparing, cells (including, for example, induced pluripotent stem cells (iPSCs)) using bioreactor-based systems, and more specifically to methods for loading and distributing cells in the bioreactor-based systems and to methods for expanding cells loaded and distributed in the bioreactor-based systems.

### BACKGROUND

This section provides background information related to the present disclosure which is not necessarily prior art.

Induced pluripotent stem cells (iPSCs) are cells that are derived from a range of terminally differentiated tissues that have been induced to a pluripotent state. Expansion of this cell type is often difficult, in particular, in the instance of bioreactor-based systems, for example, because of the tendency of the cells to attach weakly to the attachment surface or substrate and detach as a result of mechanical or sheer forces in and applied to the culture environment. It is often necessary to see the induced pluripotent stem cells onto the attachment surface or substrate in a substantially even distribution to maximize expansion potential and/or to minimize spontaneous differentiation into non-pluripotent phenotypes. Accordingly, it would be desirable to develop methods for producing, or preparing, cells (including, for example, induced pluripotent stem cells (iPSCs)) using bioreactor-based systems.

### SUMMARY

This section provides a general summary of the disclosure, and is not a comprehensive disclosure of its full scope or all of its features.

In various aspects, the present disclosure provides an example method for using a bioreactor-based system.

In at least one example embodiment, the method may include loading and distributing cells in a space of the bioreactor-based system.

In at least one example embodiment, the loading and distributing of the cells in the space of the bioreactor-based system may include circulating a volume of fluid including a cell population through the space of the bioreactor-based system in a first direction for a first period and, after the first period, circulating the volume of fluid including the cell population through the space of the bioreactor-based system in a second direction that is different from the first direction for a second period.

In at least one example embodiment, the method may further include, after the second period, circulating the volume of fluid including the cell population through the space of the bioreactor-based system in the first direction for a third period.

In at least one example embodiment, the space of the bioreactor-based system may have a first orientation during the circulating of the volume of fluid including the cell population through the space in the first direction for the first period.

In at least one example embodiment, the space of the bioreactor-based system may have a second orientation that is different from the first orientation during the circulating of the volume of the fluid including the cell population though the space in the first direction for the third period.

In at least one example embodiment, the space of the bioreactor-based system may have the first orientation during the circulating of the volume of fluid including the cell population through the space in the second direction of the second period.

In at least one example embodiment, the method may further include, after the circulating of the volume of fluid including the cell population through the space of the bioreactor-based system in the second direction of the second period, and before the circulating of the volume of fluid including the cell population through the space of the bioreactor-based system in the first direction for the third period, rotating the space of the bioreactor-based system from the first orientation to the second orientation.

In at least one example embodiment, the method may further include, after the third period, circulating the volume of fluid including the cell population through the space of the bioreactor-based system in the second direction for a fourth period.

In at least one example embodiment, the space of the bioreactor-based system may have a first orientation during the circulating of the volume of fluid including the cell population through the space in the first direction for the first period and the first orientation during the circulating of the volume of fluid including the cell population through the space in the second direction for the second period.

In at least one example embodiment, the space of the bioreactor-based system may have a second orientation different from the first orientation during the circulating of the volume of the fluid including the cell population though the space in the first direction for the third period and the second orientation during the circulating of the volume of fluid including the cell population through the space in the second direction of the fourth period.

In at least one example embodiment, the volume of fluid including the cell population may be circulated at a first rate in the first direction for the first period and the volume of fluid including the cell population may be circulated at a second rate in the first direction of the third period, where the second rate is the same as the first rate.

In at least one example embodiment, the third period may be the same length as the first period.

In at least one example embodiment, the volume of fluid including the cell population may be circulated at a third rate in the second direction for the second period and the volume of fluid including the cell population may be circulated at a fourth rate in the second direction of the fourth period, the fourth rate being the same as the third rate.

In at least one example embodiment, the fourth period may be the same length as the second period.

In at least one example embodiment, the method may further include expanding cells loaded and distributed in the space of the bioreactor-based system.

In at least one example embodiment, the expanding of the cells loaded and distributed in the space of the bioreactor-based system may include maintaining a cell culture medium in the space of the bioreactor-based system using bidirectional movement of the cell culture medium at a first rate from both a first end and an opposing second end of the space.

In at least one example embodiment, the expanding of the cells loaded and distributed in the space of the bioreactor-based system may include causing the cell culture medium to move through the space of the bioreactor-based system at a second rate that is less than the first rate.

In at least one example embodiment, the second rate may be greater than or equal to about 0.5 mL/minute to less than or equal to about 2 mL/minute.

In at least one example embodiment, the expanding of the cells loaded and distributed in the space of the bioreactor-based system may include causing a washing fluid to move through the space of the bioreactor-based system.

In various aspects, the present disclosure provides another example method for using a bioreactor-based system.

In at least one example embodiment, the method may include circulating a volume of fluid including a cell population through a space of the bioreactor-based system in a first direction for a first period, where the space has a first orientation for the first period; after the first period, circulating the volume of fluid including the cell population through the space of the bioreactor-based system in a second direction that is different from the first direction for a second period, where the space has the first orientation for the second period; after the second period, circulating the volume of fluid including the cell population through the space of the bioreactor-based system in the first direction for a third period, where the space has a second orientation different from the first orientation for the third period; and after the third period, circulating the volume of fluid including the cell population through the space of the bioreactor-based system in the second direction for a fourth period, where the space has the second orientation for the fourth period.

In at least one example embodiment, the method may further include, after the second period and before the third period, rotating the space from the first orientation to the second orientation.

In at least one example embodiment, the method may further include maintaining a cell culture medium in the space of the bioreactor-based system using bidirectional movement of the cell culture medium at a first rate from both a first end and an opposing second end of the space, causing the cell culture medium to move through the space of the bioreactor-based system at a second rate that is less than the first rate, and causing a washing fluid to move through the space of the bioreactor-based system.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is an illustration of an example cell expansion system having a bioreactor in accordance with at least one example embodiment;

FIG. 2 is an illustration of an example bioreactor that shows circulation paths through the bioreactor, and which may be incorporated into cell expansion systems, like the cell expansion system illustrated in FIG. 1, in accordance with at least one example embodiment;

FIG. 3 is an illustration of an example rocking device configured to move a bioreactor, such as the bioreactor illustrated in FIG. 2, in accordance with at least one example embodiment;

FIG. 4 is a schematic illustrating example flow paths of an example cell expansion system, like the cell expansion system illustrated in FIG. 1, in accordance with at least one example embodiment of the present disclosure;

FIG. 5 is a flowchart illustrating an example method for loading and distributing cells, like induced pluripotent stem cells (iPSCs), mesenchymal stromal cells (MSCs), or HEK293T cells, in a bioreactor, such as may be included in the cell expansion system illustrated in FIG. 1, in accordance with at least one example embodiment;

FIG. 6 is a schematic of an example counterflow scheme for a bioreactor, like the bioreactor illustrated in FIG. 2, in accordance with at least one example embodiment; and

FIG. 7 is a flow chart illustrating an example method for attaching and expanding cells, like induced pluripotent stem cells (iPSCs), loaded and distributed, for example, using the method illustrated in FIG. 5, in a bioreactor, such as may be included in the cell expansion system illustrated in FIG. 1, in accordance with at least one example embodiment.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

In this application, including the definitions below, the term “module” or the term “controller” may be replaced with the term “circuit.” The term “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

The term code, as used above, may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The term memory circuit is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only memory circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The computer programs include processor-executable instructions that are stored on at least one non-transitory, tangible computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation) (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python@.

Example embodiments will now be described more fully with reference to the accompanying drawings.

Cell expansion systems are cell culturing systems used to expand and differentiate cells, including both adherent and non-adherent cell types. The present disclosure relates to cell expansion systems and processes of preparing and using the same. In at least one example embodiment, the present disclosure relates to systems and methods for producing or preparing induced pluripotent stem cells (iPSCs) and/or other like cells using cell expansion systems. Example cell expansion systems are described, for example, in U.S. Pat. No. 11,702,634 on Jul. 18, 2023 and titled EXPANDING CELLS IN A BIOREACTOR, which was filed as U.S. application Ser. No. 15/943,536 as filed Apr. 2, 2018; U.S. Pat. No. 10,577,585 as issued on Mar. 3, 2020 and titled CELL EXPANSION, which was filed as U.S. application Ser. No. 15/153,396 on May 12, 2016; U.S. Pat. No. 11,685,883 as issued on Jun. 27, 2023 and titled METHODS AND SYSTEMS FOR COATING A CELL GROWTH SURFACE, WHICH, which was filed as U.S. application Ser. No. 15/616,635 on Jun. 7, 2017; U.S. Pat. No. 12,043,823 as issued on Jul. 23, 2024 and titled CELL CAPTURE AND EXPANSION, which was filed as U.S. application Ser. No. 17/702,658 as filed Mar. 23, 2022; U.S. Pat. No. 11,999,929 as issued Jun. 4, 2023 and titled METHODS AND SYSTEMS FOR COATING A CELL GROWTH SURFACE, which was filed as U.S. application Ser. No. 16/845,686 as filed Apr. 10, 2022; U.S. application Ser. No. 17/087,571 as filed Nov. 2, 2022 and titled CELL EXPANSION, which published as U.S. Pub. No. 2021/0047602 on Feb. 18, 2021; U.S. Pat. No. 11,702,634 as issued Jul. 18, 2023 and titled EXPANDING CELLS IN A BIOREACTOR, which was filed as U.S. application Ser. No. 15/943,536 on Apr. 2, 2018; U.S. application Ser. No. 18/234,470 as filed Aug. 16, 2023 and titled METHODS FOR CELL EXPANSION, DIFFERENTIATION, AND/OR HARVESTING OF NATURAL KILLER CELLS USING HOLLOW-FIBER MEMBRANES, which published as U.S. Pub. No. 2024/0076597 on Mar. 7, 2024; and U.S. application Ser. No. 18/368,879 as filed Sep. 15, 2023 and titled EXPANDING CELLS, which published as U.S. Pub. No. 2024/0101946 on Mar. 28, 2023, the entire disclosures of which are hereby incorporated by reference.

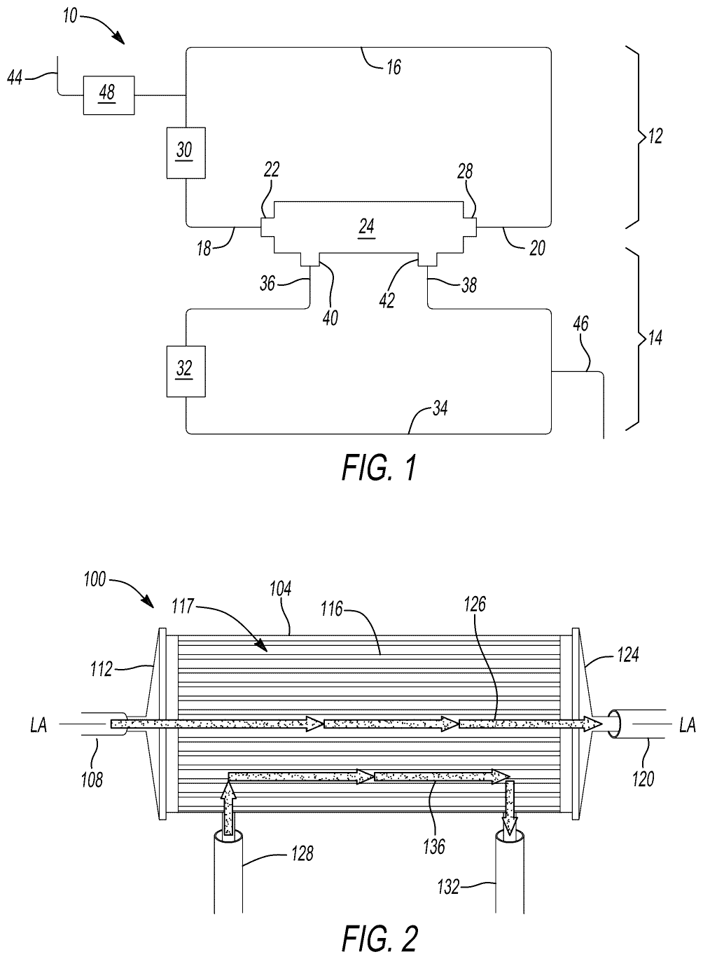

FIG. 1 is an illustration of an example cell expansion system 10. The cell expansion system 10 includes a first fluid circulation path 12 and a second fluid circulation path 14. The first fluid circulation path 12 may include, for example, a first fluid flow path 16 having opposing ends 18 and 20. The first fluid flow path 16 may be in fluid communication with a cell growth chamber 24. For example, the first opposing end 18 of the first fluid flow path 16 may be in fluid communication with a first inlet 22 of the cell growth chamber 24, and the second opposing end 20 may be in fluid communication with first outlet 28 of the cell growth chamber 24. The cell growth chamber 24 may include, or be configured to receive, a bioreactor (which may also be referred to as a hollow fiber membrane (HFM)) 117 (see FIG. 2).

The bioreactor 117 may be a standard bioreactor or a small bioreactor. The standard and small bioreactors may be similarly configured and received by the same cell expansion system 10. The standard and small bioreactors, however, have different general dimensions. A standard bioreactor may be generally selected to accommodate larger cell seeds (e.g., greater than 25 M cells) and/or to produce larger cell harvests (e.g., greater than 2 B cells), while a small bioreactor may be generally selected to accommodate smaller cell seeds (e.g., less than or equal to 25 M Cells) and/or to product smaller cell harvests (e.g., less than 3 B cells) and/or to maintain lower costs. In each instance, fluid in the first circulation path 12 may flow through an interior of a plurality of hollow fibers 116 of the bioreactor 117. In at least one example embodiment, a first fluid flow control device 30 may be operably coupled to the first fluid flow path 16 and may control the flow of fluid in first fluid circulation path 12.

The second fluid circulation path 14 may include, for example, a second fluid flow path 34 and a second fluid flow control device 32. Like the first fluid flow path 16, the second fluid flow path 34 may have opposing ends 36 and 38. The opposing ends 36 and 38 of second fluid flow path 34 may in fluid communication with an inlet port 40 and an outlet port 42 of the cell growth chamber 24. For example, a first opposing end 36 of the second fluid flow path 34 may be in fluid communication with the inlet port 40 of the cell growth chamber 24, and the second opposing end 38 of the second fluid flow path 34 may be in fluid communication with the outlet port 42. Fluid in the second circulation path 14 may be in contact with an outside of the bioreactor 117 disposed in the cell growth chamber 24. In at least one example embodiment, a second fluid flow control device 32 may be operably coupled to the second fluid flow path 34 and may control the flow of fluid in the second fluid circulation path 14.

The first and second fluid circulation paths 12, 14 may be maintained in the cell growth chamber 24 by way of the bioreactor 117, where fluid in first fluid circulation path 12 flows through an intracapillary (IC) space of the bioreactor 117 and fluid in the second circulation path 14 flows through the extracapillary (EC) space of the cell growth chamber 24. The first circulation path 12 may be referred to as the “intracapillary loop” or “IC loop”. The second fluid circulation path 14 may be referred to as the “extracapillary loop” or “EC loop”. Fluid in first fluid circulation path 12 may flow in either a co-current or counter-current direction with respect to a fluid flow in second fluid circulation path 14.

In at least one example embodiment, a fluid inlet path 44 may be fluidly associated with the first fluid circulation path 12, and a fluid outlet path 46 may be fluidly associated with the second fluid circulation path 14. The fluid inlet path 44 may permit fluid into first fluid circulation path 12, while the fluid outlet path 46 may permit fluid to exit the cell expansion system 10. In at least one example embodiment, as illustrated, a third fluid flow control device 48 may be operably associated with the fluid inlet path 44. Although not illustrated, it should be recognized that, in various other example embodiments, a fourth fluid flow control device may alternatively or additionally be operably associated with the first outlet path 46. In at least one example embodiment, the fluid flow control devices (including the first fluid flow control device 30 and/or the second fluid flow control device 32 and/or the third fluid flow control device 48 and/or the fourth fluid flow control device) may include a pump, valve, clamp, or any combination thereof. For example, multiple pumps, valves, and clamps can be arranged in any combination. In at least one example embodiment, the fluid flow control device may be, or include, a peristaltic pump. Fluid circulation paths (including the first fluid circulation path 12 and/or the second fluid circulation path 14) and/or inlet ports (including the fluid inlet port 44) and/or the outlet port (including the fluid outlet port 46) may include any known tubing material, and any kind of fluid—including, for example, buffers, protein containing fluid, and cell-containing fluid—can flow through the various circulation paths (including the first fluid circulation path 12 and/or the second fluid circulation path 14) and/or the inlet paths (including the fluid inlet port 44), and outlet paths (including the fluid outlet port 46). It should be recognized that the terms “fluid,” “media,” and “fluid media” are used interchangeably.

An example cell growth chamber 100 is illustrated in FIG. 2. The cell growth chamber 100 may be used as the cell growth chamber 24 of the cell expansion system 10 illustrated in FIG. 1. The cell growth chamber 100 may have a longitudinal axis (represented by the line LA-LA) and may include a cell growth chamber housing 104. The cell growth chamber housing 104 may have four openings or ports, including, for example, an intracapillary inlet port 108, an intracapillary outlet port 120, an extracapillary inlet port 128, and an extracapillary outlet port 132. A first fluid (which can also be referred to as an intracapillary fluid or media) in a first circulation path (like the first fluid circulation path 12) can enter the cell growth chamber 100 through the intracapillary inlet port 108 at a first fluid manifold end 112 of the cell growth chamber 100 and into and through the intracapillary spaces of a plurality of hollow fibers 116 and out of cell growth chamber 100 through intracapillary outlet port 120, which is located at a second fluid manifold end 124 of the cell growth chamber 100. The fluid path between the intracapillary inlet port 108 and the intracapillary outlet port 120 may define an intracapillary portion 126 of the cell growth chamber 100. A second fluid (which can also be referred to as an extracapillary media or fluid) in a second circulation path (like the second fluid circulation path 14) can enter the cell growth chamber 100 through the extracapillary inlet port 128. This second fluid contacts the extracapillary space or outside of the bioreactor 117 and exits the cell growth chamber 100 via the extracapillary outlet port 132. The fluid path between the extracapillary inlet port 128 and the extracapillary outlet port 132 may define an extracapillary portion 136 of the cell growth chamber 100.

As the second fluid comes into contact with the outside of the hollow fibers 116 small molecules (e.g., ions, water, oxygen, lactate, etc.) may diffuse through the hollow fibers 116 from the interior or intracapillary space of the hollow fibers 116 to the exterior or extracapillary space, or alternatively, or additionally, from the extracapillary space to the intracapillary space. Large molecular weight molecules (e.g., growth factors and/or proteins) are often too large to pass through the membrane walls of the hollow fibers 116 and remain in the intracapillary space (or alternatively, or additionally, in the extracapillary space) of the hollow fibers 116. The mediums defining the first and second fluids may be replaced as needed and may alternatively, or additionally, be circulated through an oxygenator and/or gas transfer module to exchange gasses, as needed. As discussed below, cells for expansion (e.g., induced pluripotent stem cells) may be contained within the first fluid circulation path 12 and/or the second fluid circulation path 14 and may enter the cell growth chamber 100 on one or both of the intracapillary space or the extracapillary space.

Often, cells for expansion (including, for example, induced pluripotent stem cells) are seeded (for example, for expansion, differentiation, and/or harvesting of mesenchymal stromal cells from bone marrow, adipose tissue, or umbilical cord blood, HEK293T cells, T-cells, NK cells, muscle satellite cells, cord blood derived CD34+ hematopoietic stem/progenitor cells, monocytes, macrophages, hepatocytes, and/or endothelial cells) in the intracapillary space 130, while a cell culture medium is pumped through the extracapillary space 110 to deliver nutrients to the cells via hollow-fiber membrane perfusion during expansion. However, in other variations, cells for expansion (including, for example, induced pluripotent stem cells) can be seeded (for example, for expansion, differentiation, and/or harvesting of mesenchymal stromal cells from bone marrow, adipose tissue, or umbilical cord blood, HEK293T cells, T-cells, NK cells, muscle satellite cells, cord blood derived CD34+ hematopoietic stem/progenitor cells, monocytes, macrophages, hepatocytes, and/or endothelial cells) in the extracapillary space 110, while the cell culture medium is pumped through the intracapillary space 130 to deliver nutrients to the cells via hollow-fiber membrane perfusion during expansion. In still further variations, cells for expansion (including, for example, induced pluripotent stem cells) may be seeded (for example, for expansion, differentiation, and/or harvesting of mesenchymal stromal cells from bone marrow, adipose tissue, or umbilical cord blood, HEK293T cells, T-cells, NK cells, muscle satellite cells, cord blood derived CD34+ hematopoietic stem/progenitor cells, monocytes, macrophages, hepatocytes, and/or endothelial cells) in the intracapillary space 130, while the cell culture medium is pumped through both the extracapillary space 110 and the intracapillary space 130. In such instances, movement of the cell culture medium through the intracapillary space 130 can help to remove excess cells not adhered to surfaces of the hollow-fiber membrane 101. In each instance, the material used to form the hollow-fiber membrane 101 may be any biocompatible polymeric material that is capable of being made into the hollow fibers 121. For example, synthetic polysulfone-based materials (e.g., polyethersulfones (PES)) are often used to form the hollow fibers.

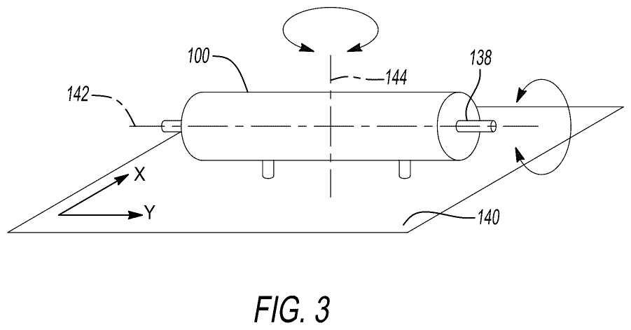

In at least one example embodiment, the cell expansion system 10 may also include a device that is configured to move or “rock” the cell growth chamber 100 relative to other components of the cell expansion system 10. The device may be a rotational and/or lateral rocking device. For example, as illustrated in FIG. 3, the cell growth chamber 100 may be rotationally connected to one or more rotational rocking components 138 and to a lateral rocking component 140. A first rotational rocking component 138 may be rotationally associated with the cell growth chamber 100. For example, the first rotational rocking component 138 may be configured to rotate the cell growth chamber 100 around a first or central rotational axis 142. In at least one example embodiment, the cell growth chamber 100 may be rotated in alternating fashion, including, for example, in a first clockwise direction and then in a second counterclockwise direction around the central axis 142.

Although not illustrated, it should be recognized that in at least one example embodiment, a second rotational rocking component may be configured to move the cell growth chamber 100 about a second rotational axis 144 that passes through a center point of the cell growth chamber 100 normal to the central axis 142. In at least one example embodiment, the cell growth chamber 100 may be rotated in alternating fashion, including, for example, in a first clockwise direction and then in a second counterclockwise direction around the second axis 144. In at least one example embodiment, the cell growth chamber 100 may also be rotated around the second axis 144 and positioned in a horizontal or vertical orientation relative to gravity. The lateral rocking component 140 may be laterally associated with the cell growth chamber 100. For example, a plane of the lateral rocking component 140 may move laterally in the x-direction and y-direction.

The rotational and/or lateral movement of the cell growth chamber 100 may reduce the settling of cells and the likelihood of cells becoming trapped within a portion of the bioreactor 117 disposed in the cell growth chamber 100. In at least one example embodiment, the rate of cells settling in the cell growth chamber 100 may be proportional to the density difference between the cells and the suspension media, according to Stoke's Law. In at least one example embodiment, a 180-degree rotation (fast) with a pause (having, for example, a total combined time of 30 seconds) repeated as described above may help to keep non-adherent cells (for example, T-cells) suspended. A minimum rotation of about 180-degrees may be preferred, however various degrees of rotation, including up to or greater than 360-degrees, may be used. Different rocking components may be used separately or may be combined in any combination. For example, a rocking component that rotates cell growth chamber 100 around central axis 142 may be combined with the rocking component that rotates cell growth chamber 100 around axis 144. Likewise, clockwise and counterclockwise rotation around different axes may be performed independently in any combination.

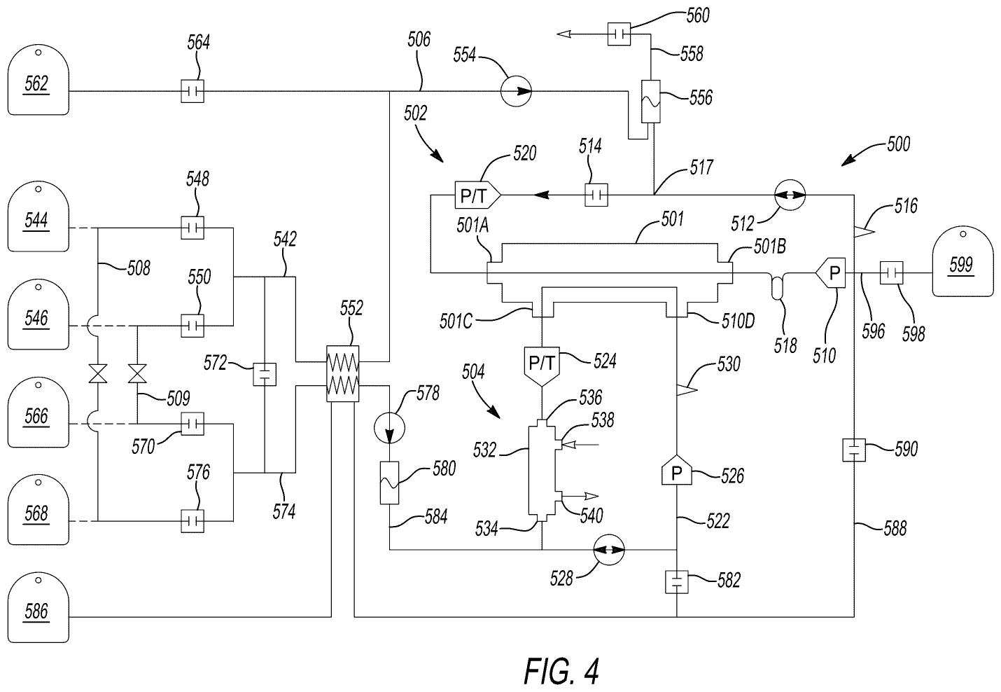

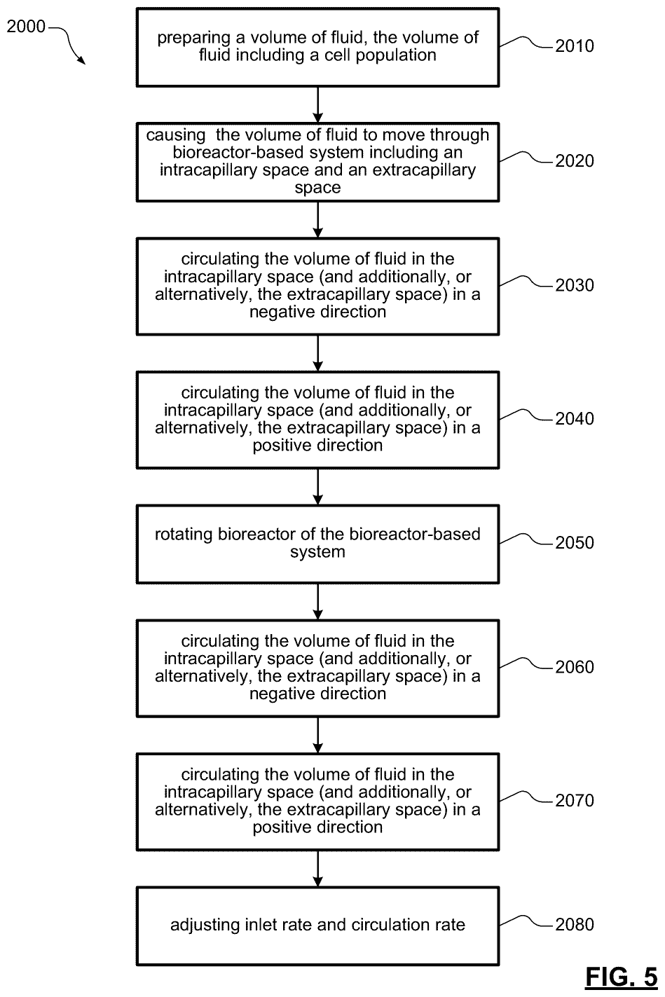

FIG. 4 is a schematic of an example cell expansion system 500, which may be like the cell expansion system 100 illustrated in FIG. 1, that illustrates example flow paths. In at least one example embodiment, the cells may be positioned in the intracapillary space, while a cell culture medium may be pumped through the extracapillary space to deliver nutrients to the cells via hollow fiber membrane perfusion during expansion. It should be recognized, however, in at least one other example embodiment, cells can be positioned in the extracapillary space, while the cell culture medium may be pumped through the intracapillary space to deliver nutrients to the cells via hollow fiber membrane perfusion during expansion. In at least one other example embodiment, cells may be positioned in the intracapillary space, while the cell culture medium may be pumped through both the extracapillary space and the intracapillary space.

As illustrated, the cell expansion system 500 may include a first fluid circulation path 502 (also referred to as the “intracapillary loop” or “IC loop”) and a second fluid circulation path 504 (also referred to as the “extracapillary loop” or “EC loop”). The first fluid flow path 506 may be fluidly associated with a cell growth chamber 501 to form first fluid circulation path 502. The cell growth chamber 501 may be used as the cell growth chamber 24 illustrated in FIG. 1 and/or the cell growth chamber 100 illustrated in FIG. 2. A first fluid may flow into cell growth chamber 501 through an intracapillary inlet port 501A. The first fluid may exit the cell growth chamber via an intracapillary outlet port 501B. In at least one example embodiment, the first fluid circulation path 502 may include a pressure gauge 510 configured to measure a pressure of the first fluid leaving the cell growth chamber 501. In at least one example embodiment, the first fluid circulation path 502 may include an intracapillary circulation pump 512 configured to control a first fluid flow rate. For example, the intracapillary circulation pump 512 may be configured to pump the first fluid in a first direction or a second direction that is opposite to the first direction. In the later instance, the intracapillary outlet port 501B may be used as an inlet, and the intracapillary inlet port 501A as an outlet. In at least one example embodiment, the first fluid circulation path 502 may include a sample port 516 and/or sample coil 518 configured for first fluid sample extraction. In at least one example embodiment, the first fluid circulation path 502 may include a pressure/temperature gauge 520 configured to detect the pressure and/or temperature of the first fluid during operation. In at least one example embodiment, the first fluid may enter the intracapillary loop 502 via valve 514. In at least one example embodiment, a portion of the cells may be flushed from the intracapillary loop 502 into a harvest bag 599, for example, via valve 598. It should be recognized that, in at least one other example embodiment, the first fluid circulation path 502 may include additional or fewer valves, pressure gauges, pressure sensors, temperature sensors, ports, and/or other devices disposed to isolate and/or measure characteristics of the first fluid along portions of the intracapillary loop 502.

A second fluid may flow into cell growth chamber 501 through an extracapillary inlet port 501C. The second fluid may leave the cell growth chamber 501 via an extracapillary outlet port 501D. In at least one example embodiment, the second fluid in the extracapillary loop 504 may contact an exterior facing surface of hollow fibers disposed in the cell growth chamber 501 thereby allowing diffusion of small molecules into and out of the hollow fibers. In at least one example embodiment, the extracapillary loop 504 may include a pressure/temperature gauge 524 configured to measure a pressure and/or temperature of the second fluid before the second fluid enters the cell growth chamber 501. In at least one example embodiment, the extracapillary loop 504 may include a pressure gauge 526 that is configured to measure a pressure of the second fluid, for example, as it leaves the cell growth chamber 501. In at least one example embodiment, the extracapillary loop 504 may include a sample port 530 configured for second fluid sample extraction.

In at least one example embodiment, the extracapillary loop 504 may include an extracapillary circulation pump 528 and an oxygenator or gas transfer module 532. For example, after leaving the cell growth chamber 501, the second fluid may pass through the extracapillary circulation pump 528 and to and through the oxygenator or gas transfer module 532. In at least one example embodiment, the extracapillary circulation pump 528 may be configured to control a second fluid flow rate. For example, like the intracapillary circulation pump 512, the extracapillary circulation pump 528 may be configured to pump the second fluid in a first direction or a second direction that is opposite to the first direction. In the later instance, the extracapillary outlet port 501D may be used as inlet, and the extracapillary inlet port 501C as an outlet.

In at least one example embodiment, the second fluid flow path 522 may be fluidly associated with the oxygenator or gas transfer module 532 via an oxygenator inlet port 534 and an oxygenator outlet port 536. For example, the second fluid may flow into the oxygenator or gas transfer module 532 via the oxygenator inlet port 534 and may leave or exit the oxygenator or gas transfer module 532 via the oxygenator outlet port 536. In at least one example embodiment, the oxygenator or gas transfer module 532 may be configured to add oxygen to and/or remove bubbles from the second fluid. For example, air and/or gas may flow into the oxygenator or gas transfer module 532 via a first filter 538 and may leave or exit (i.e., flow out of) the oxygenator or gas transfer device 532 through a second filter 540. The first and second filters 538, 540 may be configured to reduce or prevent contaminants from entering the oxygenator or gas transfer module 532. The second fluid in the second fluid circulation path 504 may be in equilibrium with gas entering the oxygenator or gas transfer module 532. In at least one example embodiment, air and/or gas may be purged from the cell expansion system 500, for example, during a priming sequence, air and/or gas may be vented to the atmosphere via the oxygenator or gas transfer module 532. It should be recognized that, in at least one other example embodiment, a second fluid circulation path 504 may include additional or fewer valves, pressure gauges, pressure sensors, temperature sensors, ports, and/or other devices disposed to isolate and/or measure characteristics of the second fluid along portions of the extracapillary loop 504.

In at least one example embodiment, an air removal chamber (ARC) 556 may be fluidly associated with the first circulation path 502. The air removal chamber 556 may include one or more ultrasonic sensors. For example, the air removal chamber 556 may include upper sensor and/or lower sensor which are configured to detect air and/or a lack of fluid and/or gas-fluid interface at certain measuring positions within the air removal chamber 556. The upper sensor may be disposed near a first end (e.g., top) of the air removal chamber 556. The lower sensor may be disposed near a second end (e.g., bottom) of the air removal chamber 556. Although ultrasonic sensors are discussed, it should be appreciated that the air removal chamber 556 may include, additionally, or alternatively, one or more other sensors, including, for example, optical sensors. Air and/or gas purged from the cell expansion system 500 during portions of a priming sequence and/or other protocols may vent to the atmosphere out air valve 560 via line 558 that may be fluidly associated with air removal chamber 556.

In at least one example embodiment, the first fluid may include cells (for example, from a first fluid container (which can also be referred to as a first media bag or a first bag) 562 and also fluid media (e.g., intracapillary media or fluid) from a second fluid container (which can also be referred to as a second media bag or a second bag) 546. Materials (i.e., cells and/or intracapillary media) form the first and second fluid containers 562, 546 may enter the first fluid circulation path 502 via a first fluid flow path 506. The first fluid container 562 may be fluidly associated with the first fluid flow path 506 and the first fluid circulation path 502 via valve 564. In at least one example embodiment, the second fluid container 546 and a third fluid container (which can also be referred to as a third media bag or third bag) 544 may be fluidly associated with the first fluid inlet path 542, for example, via valves 548 and 550, respectively, or with a second fluid inlet path 574, for example, via valves 570 and 576, respectively. In at least one example embodiment, the materials from the second fluid container 546 and/or the third fluid container 544 may be in fluid communication with a first sterile sealable input priming path 508 and/or a second sterile sealable input priming path 509.

In at least one example embodiment, a fourth fluid container (which can also be referred to as a fourth media bag or a fourth bag) 568 may include an extracapillary media, and a fifth fluid container (which can also be referred to a fifth media bag or a fifth bag) 566 may include a wash solution. Materials (i.e., extracapillary media and/or wash solution) from the fourth and fifth fluid containers 568, 566 may enter the first fluid circulation path 502 and/or the second fluid circulation path 504. For example, in at least one example embodiment, the fifth fluid container 566 may be fluidly associated with valve 570, where valve 570 is fluidly associated with first fluid circulation path 502, for example, via a distribution valve 572 and a first fluid inlet path 542. In at least one example embodiment, the fifth fluid container 566 may be fluidly associated with the second fluid circulation path 504 via the second fluid inlet path 574 and an extracapillary inlet path 584, for example, by opening valve 570 and closing distribution valve 572. The fourth fluid container 568 may be fluidly associated with valve 576, where valve 576 is fluidly associated with first fluid circulation path 502, for example, via the first fluid inlet path 542 and the distribution valve 572. In at least one example embodiment, the fourth fluid container 568 may be fluidly associated with the second fluid inlet path 574 by opening valve 576 and closing the distribution valve 572. In at least one example embodiment, the first fluid inlet path 542 and/or the second fluid inlet path 574 may be fluidly associated with an optional heat exchanger 552.

In at least one example embodiment, fluid may be advanced to the intracapillary loop 502 from the first fluid inlet path 542 and/or the second fluid inlet path 574 via an intracapillary inlet pump 554, and fluid may be advanced to the extracapillary loop 504 via an extracapillary inlet pump 578. In at least one example embodiment, an air detector 580 may also be associated with the extracapillary inlet path 584. The air detector 580 may include, for example, an ultrasonic sensor. In at least one example embodiment, the first and second fluid circulation paths 502, 504 may be fluidly associated with a waste line 588. For example, when valve 590 is in an open state or position, the intracapillary media may flow through the waste line 588 to a waste bag (also referred to as an outlet bag) 586. When valve 582 is opened, extracapillary media may flow through the waste line 588 to the waste bag 586. In at least one example embodiment, cells may be harvested, for example, via a cell harvest path 596. For example, cells from the cell growth chamber 501 may be harvested by pumping the intracapillary media containing the cells through the cell harvest path 596 and also valve 598 to a cell harvest bag 599.

In at least one example embodiment, as illustrated, the fluid in the first fluid circulation path 502 and second fluid circulation path 504 flows through cell growth chamber 501 in the same direction (i.e., a co-current configuration). Although not illustrated, it should be recognized that, in various other example embodiments, the cell expansion system 500 may also be configured to flow in a counter-current conformation. As illustrated in FIG. 4, fluid in the first fluid circulation path 502 may enter the cell growth chamber 501 at the intracapillary inlet port 501A and may leave or exit the cell growth chamber 501 at the intracapillary outlet port 501B. In at least one example embodiment, the first fluid flow path 506 may be fluidly connected to the first fluid circulation path 502, for example, via connection 517. Connection 517 may be a point or location from which the fluid may flow in opposite directions, for example, based on the direction and flow rates of the intracapillary inlet pump 554 and fluid circulation pump 512. Connection 517 may include any type of fitting, coupling, fusion, pathway, and/or tubing that allows the first fluid flow path 506 to be fluidly associated with the first fluid circulation path 502. In at least one example embodiment, connection 517 may include a T-fitting or coupling and/or a Y-fitting or coupling.

In at least one example embodiment, one or more of the gauges (e.g., pressure gauge 510 and/or pressure/temperature gauge 520 and/or pressure/temperature gauge 524 and/or pressure gauge 526), one or more of the valves (e.g., valve 514 and/or valves 548 and/or valves 550 and/or valve 560 and/or valve 564 and/or valve 570 and/or valve 572 and/or valve 576 and/or valve 582 and/or valve 590 and/or valve 596 and/or valve 598), one or more of the ports (e.g., intracapillary inlet port 501A and/or intracapillary outlet port 501B and/or extracapillary inlet port 501C and/or extracapillary outlet port 501D and/or sample port 516 and/or sample port 530 and/or oxygenator inlet port 534 and/or an oxygenator outlet port 536), one or more of the pumps (e.g., intracapillary circulation pump 512 and/or extracapillary circulation pump 528 and/or intracapillary inlet pump 554 and/or extracapillary inlet pump 578), one or more of the filters (e.g., first filter 538 and/or second filter 540), one or more coils (e.g., sample coil 518), one or more modules (e.g., oxygenator or gas transfer module 532), and/or one or more other components of the cell expansion system 500 may be in electrical communication with a control system (not shown). The control system may include a plurality of nodes, which can include various hardware, firmware, and/or software configured to control and/or communicate with the mechanical, electromechanical, and electrical components of the cell expansion system 500, including for example, a controller and a memory.

The controller (which can also be referred to as a processor), can be of any type of microcontroller, microprocessor, Field Programmable Gate Array (FPGA), Application Specific Integrated Circuit (ASIC), etc. An example controller may be the NK10DN512VOK10 microcontroller, made and sold by N9P USA, Incorporated, which is a microcontroller unit with a 32-bit architecture. Other examples controllers may include, for example, at least one of Qualcomm® Snapdragon® 800 and 801, Qualcomm® Snapdragon®610 and 615 with 4G LTE Integration and 64-bit computing, Apple® A7 processor with 64-bit architecture, Apple® M7 motion coprocessors, Samsung® Exynos® series, the Intel® Core™ family of processors, the Intel® Xeon® family of processors, the Intel® Atom™ family of processors, the Intel Itanium® family of processors, Intel® Core® i5-4670K and i7-4770K 22 nm Haswell, Intel® Core® i5-3570K 22 nm Ivy Bridge, the AMD® FX™ family of processors, AMD® FX-4300, FX-6300, and FX-8350 32 nm Vishera, AMD® Kaveri processors, ARM® Cortex™-M processors, ARM® Cortex-A and ARM926EJ-S™ processors, other industry-equivalent processors, and may perform computational functions using any known or future-developed standard, instruction set, libraries, and/or architecture. The memory can be any type of memory including random access memory (RAM), read only memory (ROM), electrically erasable programmable ROM (EEPROM), a portable compact disc read-only memory (CD-ROM), an optical storage device, a magnetic storage device, any suitable combination of the foregoing, or other type of storage or memory device that stores and provides instructions to program and control the controller.

In various aspects, the present disclosure provides methods for preparing bioreactor-based systems (such as, the cell expansion system 10 illustrated in FIG. 1 and/or the bioreactor illustrated in FIG. 2 and/or the bioreactor 501 illustrated in FIG. 4) for cell expansion and/or differentiation and/or harvesting. Methods for preparing bioreactor-based systems may include, for example, coating at least a portion of the bioreactor-based systems, and more specifically, at least a portion of the intracapillary spaces, and additionally, or alternatively, the extracapillary spaces, of a bioreactor of the bioreactor-based system. The coating may help to improve expansion and/or differentiation and/or harvesting of select cells (such as induced pluripotent stem cells). In at least one example embodiment, the bioreactor-based systems, and more specifically, at least a portion of the intracapillary spaces, and additionally, or alternatively, the extracapillary spaces, of the bioreactor-based systems, may be coated with recombinant laminin, recombinant vitronectin, or a combination thereof. In at least one example embodiment, the intracapillary space, and additionally, or alternatively, the extracapillary space, may be coated using an active coating method, such as detailed, for example, in U.S. application Ser. No. 18/368,879 as filed Sep. 15, 2023 and titled EXPANDING CELLS, which published as U.S. Pub. No. 2024/0101946 on Mar. 28, 2024, and/or U.S. App. No. 63/538,610 as filed Sep. 15, 2023 and titled SYSTEMS AND METHODS FOR PRODUCING CHIMERIC ANTIGEN RECEPTOR CELLS and/or U.S. App. No. 63/461,989 as filed Apr. 26, 2023 and titled METHODS TO REMOVE SOLUTES WITHOUT SUSPENSION CELL LOSS and/or U.S. Pat. No. 11,702,634 as issued Jul. 18, 2023 and titled EXPANDING CELLS IN A BIOREACTOR, which was filed as U.S. application Ser. No. 15/943,536 on Apr. 2, 2018, the entire disclosures of which are hereby incorporated by reference.

In various aspects, the present disclosure provides methods for loading and distributing cells (like induced pluripotent stem cells) in bioreactor-based systems (such as, the cell expansion system 10 illustrated in FIG. 1 and/or the bioreactor illustrated in FIG. 2 and/or the bioreactor 501 illustrated in FIG. 4) for cell expansion and/or differentiation and/or harvesting. FIG. 5 illustrates an example multi-step, bidirectional flow method 2000 for loading and distributing cells (like induced pluripotent stem cells (iPSCs), mesenchymal stromal cells (MSCs), or HEK293T cells) in a bioreactor-based system systems (such as, the cell expansion system 10 illustrated in FIG. 1 and/or the bioreactor illustrated in FIG. 2 and/or the bioreactor 501 illustrated in FIG. 4) for cell expansion and/or differentiation and/or harvesting. The cells that may be loaded and distributed in the bioreactor-based system may be adherent cells or non-adherent cells in different example embodiments. Method 2000 provides multiple attachment opportunities for the cells to help improve load and distribution.

The method 2000 includes circulating 2030 a volume of fluid including a cell population (including, for example, induced pluripotent stem cells) through at least a portion of the bioreactor-based system (and more specifically, the intracapillary space, and additionally, or alternatively, the extracapillary space) in a first direction at a first rate. As used herein, the term “circulating” describes a process of moving the volume of fluid including the cell population back and forth along the intracapillary space of the bioreactor-based system. The process of circulating the volume of fluid including the cell population is contained within a bioreactor portion of the bioreactor-based system to avoid cell-loss in non-bioreactor portions of the bioreactor-based system. Circulating the volume of fluid including the cell population allows cells of the cell population to attach to different portions of fibers of the bioreactor-based system and eliminates movements of the volume of fluid including the cell population throughout an entirety of the bioreactor-based system.

In at least one example embodiment, for example, when the volume of fluid including the cell population is flowed through the intracapillary space, the first direction may be a negative direction defined between the outlet of the intracapillary space (e.g., the first outlet 28 as illustrated in FIG. 1 and/or the intracapillary outlet port 120 as illustrated in FIG. 2 and/or intracapillary outlet port 501B illustrated in FIG. 4) and the inlet of the intracapillary space (e.g., the first inlet 22 as illustrated in FIG. 1 and/or the intracapillary inlet port 108 and/or intracapillary inlet port 501A illustrated in FIG. 4). In at least one example embodiment, the first rate may be greater than or equal to about −7 mL/minute (e.g., greater than or equal to about −6 mL/minute, greater than or equal to about −5 mL/minute, or greater than or equal to about −4 mL/minute). In at least one example embodiment, the first rate may be less than or equal to about −3 mL/minute (e.g., less than or equal to about −4 mL/minute, less than or equal to about −5 mL/minute, or less than or equal to about −6 mL/minute). In at least one example embodiment, the first rate may be greater than or equal to about −7 mL/minute to less than or equal to about −3 mL/minute. For example, the first rate may be about −5 mL/minute.

The circulation 2030 of the volume of fluid including the cell population through at least a portion of the bioreactor-based system (and more specifically, the intracapillary space, and additionally, or alternatively, the extracapillary space) may occur for a first period. In at least one example embodiment, the first period may be greater than or equal to about 1 minute (e.g., greater than or equal to about 70 seconds, greater than or equal to about 80 seconds, greater than or equal to about 90 seconds, greater than or equal to about 100 seconds, greater than or equal to about 110 seconds, greater than or equal to about 120 seconds, greater than or equal to about 130 seconds, or greater than or equal to about 140 seconds). In at least one example embodiment, the first period may be less than or equal to about 2.5 minutes (e.g., less than or equal to about 140 seconds, less than or equal to about 130 seconds, less than or equal to about 120 seconds, less than or equal to about 110 seconds, less than or equal to about 100 seconds, less than or equal to about 90 seconds, less than or equal to about 80 seconds, or less than or equal to about 70 seconds). In at least one example embodiment, the first period may be greater than or equal to about 1 minute to less than or equal to about 2.5 minutes. For example, the first period may be about 2 minutes (or 120 seconds). The first period may be directly proportional to the first rate. For example, if it is the goal to move about 7.5 milliliters of fluid each round or cycle, a faster rate would induce a lower time period and a slower rate would induce a longer time period.

The method 2000 may further include circulating 2040 the volume of fluid including the cell population through at least a portion of the bioreactor-based system (and more specifically, the intracapillary space, and additionally, or alternatively, the extracapillary space) in a second direction at a second rate, where the second direction is different than the first direction. In at least one example embodiment, the circulation 2040 of the volume of fluid through at least a portion the bioreactor-based system in the second direction at the second rate may occur after the circulating 2030 of the volume of fluid through at least a portion of the bioreactor-based system in the first direction at the first rate.

In at least one example embodiment, for example, when the volume of fluid including the cell population is flowed through the intracapillary space, the second direction may be a positive direction defined between the inlet of the intracapillary space (e.g., the first inlet 22 as illustrated in FIG. 1 and/or the intracapillary inlet port 108 and/or intracapillary inlet port 501A illustrated in FIG. 4) and the outlet of the intracapillary space (e.g., the first outlet 28 as illustrated in FIG. 1 and/or the intracapillary outlet port 120 as illustrated in FIG. 2 and/or intracapillary outlet port 501B illustrated in FIG. 4). In at least one example embodiment, the second rate may be greater than or equal to about +3 mL/minute (e.g., greater than or equal to about +4 mL/minute, greater than or equal to about +5 mL/minute, or greater than or equal to about +6 mL/minute). In at least one example embodiment, the second rate may be less than or equal to about +7 mL/minute (e.g., less than or equal to about +6 mL/minute, less than or equal to about +5 mL/minute, or less than or equal to about +4 mL/minute). In at least one example embodiment, the second rate may be greater than or equal to about +3 mL/minute to less than or equal to about +7 mL/minute For example, the second rate may be about +5 mL/minute.

The circulation 2040 of the volume of fluid including the cell population through at least a portion of the bioreactor-based system may occur for a second period. The second period may be the same as or different from the first period. In at least one example embodiment, the second period may be greater than or equal to about 1 minute (e.g., greater than or equal to about 70 seconds, greater than or equal to about 80 seconds, greater than or equal to about 90 seconds, greater than or equal to about 100 seconds, greater than or equal to about 110 seconds, greater than or equal to about 120 seconds, greater than or equal to about 130 seconds, or greater than or equal to about 140 seconds). In at least one example embodiment, the second period may be less than or equal to about 2.5 minutes (e.g., less than or equal to about 140 seconds, less than or equal to about 130 seconds, less than or equal to about 120 seconds, less than or equal to about 110 seconds, less than or equal to about 100 seconds, less than or equal to about 90 seconds, less than or equal to about 80 seconds, or less than or equal to about 70 seconds). In at least one example embodiment, the second period may be greater than or equal to about 1 minute to less than or equal to about 2.5 minutes. For example, the second period may be about 1.5 minutes (i.e., 90 seconds). The second period may be directly proportional to the second rate. For example, if it is the goal to move about 7.5 milliliters of fluid each round, a faster rate would induce a lower time period and a slower rate would induce a longer time period.

In at least one example embodiment, the bioreactor-based system may have a first horizontal orientation during the circulation 2030 of the volume of fluid including the cell population in the first direction at the first rate and also during the circulation 2040 of the volume of fluid including the cell population in the second direction at the second rate. For example, the bioreactor-based system may be orientated at about 180 degrees during the circulation 2030 of the volume of fluid including the cell population in the first direction at the first rate and also during the circulation 2040 of the volume of fluid including the cell population in the second direction at the second rate. The method 2000 may include rotating 2050 the bioreactor-based system (for example, using the rocking device detailed with respect to FIG. 3 above) to a second horizontal orientation. The rotation 2050 of the bioreactor-based system may help to ensure that all available surface area is available for attachment to, and attaches to, the cell population. In at least one example embodiment, the rotation 2050 may include rotating the bioreactor-based system to about 0 degrees.

The method 2000 may include circulating 2060 of the volume of fluid including the cell population through at least a portion of the bioreactor-based system (and more specifically, the intracapillary space, and additionally, or alternatively, the extracapillary space) in the first direction at a third rate. In at least one example embodiment, the circulation 2060 of the volume of fluid through at least a portion of the bioreactor-based system in the first direction at the third rate may occur after the circulation 2040 of the volume of fluid through at least a portion of the bioreactor-based system in the second direction at the second rate. In at least one example embodiment, the circulation 2060 of the volume of fluid through at least a portion of the bioreactor-based system in the first direction at the third rate may occur after the rotation 2050 of the bioreactor-based system. In at least one example embodiment, the circulation 2060 of the volume of fluid through at least a portion of the bioreactor-based system in the first direction at the third rate may occur after the circulation 2040 of the volume of fluid through at least a portion of the bioreactor-based system in the second direction at the second rate. In at least one example embodiment, the circulation 2060 of the volume of fluid through the bioreactor-based system in the first direction at the third rate may occur after the circulating 2030 of the volume of fluid through at least a portion of the bioreactor-based system in the first direction at the first rate.

The third rate may be the same as or different from the first rate. In at least one example embodiment, the third rate may be greater than or equal to about −7 mL/minute (e.g., greater than or equal to about −6 mL/minute, greater than or equal to about −5 mL/minute, or greater than or equal to about −4 mL/minute). In at least one example embodiment, the third rate may be less than or equal to about −3 mL/minute (e.g., less than or equal to about −4 mL/minute, less than or equal to about −5 mL/minute, or less than or equal to about −6 mL/minute). In at least one example embodiment, the third rate may be greater than or equal to about −7 mL/minute to less than or equal to about −3 mL/minute. For example, in at least one example embodiment, the third rate may be about −5 mL/minute.

The circulation 2060 of the volume of fluid including the cell population through at least a portion of the bioreactor-based system may occur for a third period. The third period may be the same as or different from the first period. The third period may be the same as or different from the second period. In at least one example embodiment, the third period may be greater than or equal to about 1 minute (e.g., greater than or equal to about 70 seconds, greater than or equal to about 80 seconds, greater than or equal to about 90 seconds, greater than or equal to about 100 seconds, greater than or equal to about 110 seconds, greater than or equal to about 120 seconds, greater than or equal to about 130 seconds, or greater than or equal to about 140 seconds). In at least one example embodiment, the third period may be less than or equal to about 2.5 minutes (e.g., less than or equal to about 140 seconds, less than or equal to about 130 seconds, less than or equal to about 120 seconds, less than or equal to about 110 seconds, less than or equal to about 100 seconds, less than or equal to about 90 seconds, less than or equal to about 80 seconds, or less than or equal to about 70 seconds). In at least one example embodiment, the third period may be greater than or equal to about 1 minute to less than or equal to about 2.5 minutes. For example, the third period may be about 1.5 minutes (or 90 seconds). The third period may be directly proportional to the third rate. For example, if it is the goal to move about 7.5 milliliters of fluid each round, a faster rate would induce a lower time period and a slower rate would induce a longer time period.

The method 200 may include circulating 2070 the volume of fluid including the cell population through at least a portion of the bioreactor-based system (and more specifically, the intracapillary space, and additionally, or alternatively, the extracapillary space) in the second direction at a fourth rate. In at least one example embodiment, the circulation 2070 of the volume of fluid through at least a portion of the bioreactor-based system in the second direction at the fourth rate may occur after the circulation 2060 of the volume of fluid through at least a portion of the bioreactor-based system in the first direction at the third rate. In at least one example embodiment, the circulation 2070 of the volume of fluid through at least a portion of the bioreactor-based system in the second direction at the fourth rate may occur after the rotation 2050 of the bioreactor-based system. In at least one example embodiment, the circulation 2070 of the volume of fluid through at least a portion of the bioreactor-based system in the second direction at the fourth rate may occur after the circulation 2040 of the volume of fluid through at least a portion of the bioreactor-based system in the second direction at the second rate. In at least one example embodiment, the circulation 2070 of the volume of fluid through at least a portion of the bioreactor-based system in the second direction at the fourth rate may occur after the circulating 2030 of the volume of fluid through at least a portion of the bioreactor-based system in the first direction at the first rate.

The fourth rate may be the same as or different from the second rate. In at least one example embodiment, the fourth rate may be greater than or equal to about +3 mL/minute (e.g., greater than or equal to about +4 mL/minute, greater than or equal to about +5 mL/minute, or greater than or equal to about +6 mL/minute). In at least one example embodiment, the fourth rate may be less than or equal to about +7 mL/minute (e.g., less than or equal to about +6 mL/minute, less than or equal to about +5 mL/minute, or less than or equal to about +4 mL/minute). In at least one example embodiment, the fourth rate may be greater than or equal to about +3 mL/minute to less than or equal to about +7 mL/minute. For example, the fourth rate may be about +5 mL/minute.

The circulation 2070 of the volume of fluid including the cell population through at least a portion of the bioreactor-based system may occur for a fourth period. The fourth period may be the same as or different from the first period. The fourth period may be the same as or different from the second period. The fourth period may be the same as or different from the third period. In at least one example embodiment, the fourth period may be greater than or equal to about 1 minute (e.g., greater than or equal to about 70 seconds, greater than or equal to about 80 seconds, greater than or equal to about 90 seconds, greater than or equal to about 100 seconds, greater than or equal to about 110 seconds, greater than or equal to about 120 seconds, greater than or equal to about 130 seconds, or greater than or equal to about 140 seconds). In at least one example embodiment, the fourth period may be less than or equal to about 2.5 minutes (e.g., less than or equal to about 140 seconds, less than or equal to about 130 seconds, less than or equal to about 120 seconds, less than or equal to about 110 seconds, less than or equal to about 100 seconds, less than or equal to about 90 seconds, less than or equal to about 80 seconds, or less than or equal to about 70 seconds). In at least one example embodiment, the fourth period may be greater than or equal to about 1 minute to less than or equal to about 2.5 minutes. For example, the fourth period may be about 1.5 minutes (or 90 seconds). The fourth period may be directly proportional to the fourth rate. For example, if it is the goal to move about 7.5 milliliters of fluid each round, a faster rate would induce a lower time period and a slower rate would induce a longer time period.

The method 2000 includes adjusting 2080 the inlet rate (e.g., the intracapillary inlet rate and/or the extracapillary inlet rate) and/or the circulation rate (e.g., the intracapillary circulation rate and/or the extracapillary circulation rate). In at least one example embodiment, adjusting 2080 the inlet rate may help move any cells that are outside of the fibers into a fiber and/or to begin the process of driving suspended cells towards the (optionally coated) membrane where attachment occurs, such as discussed below. In at least one example embodiment, the adjusting 2080 of the inlet rate and/or the circulation rate may occur after the circulation 2070 of the volume of fluid through at least a portion of the bioreactor-based system in the second direction at the fourth rate. In at least one example embodiment, the adjusting 2080 of the inlet rate and/or the circulation rate may occur after the circulation 2060 of the volume of fluid through at least a portion of the bioreactor-based system in the first direction at the third rate. In at least one example embodiment, the adjusting 2080 of the inlet rate and/or the circulation rate may occur after the rotation 2050 of the bioreactor-based system. In at least one example embodiment, the adjusting 2080 of the inlet rate and/or the circulation rate may occur after the circulation 2040 of the volume of fluid through at least a portion of the bioreactor-based system in the second direction at the second rate. In at least one example embodiment, the adjusting 2080 of the inlet rate and/or the circulation rate may occur after the circulating 2030 of the volume of fluid through at least a portion of the bioreactor-based system in the first direction at the first rate.

In at least one example embodiment, the inlet rate may be greater than or equal to about +3 mL/minute (e.g., greater than or equal to about +4 mL/minute, greater than or equal to about +5 mL/minute, or greater than or equal to about +6 mL/minute). In at least one example embodiment, the inlet rate may be less than or equal to about +7 mL/minute (e.g., less than or equal to about +6 mL/minute, less than or equal to about +5 mL/minute, or less than or equal to about +4 mL/minute). In at least one example embodiment, the inlet rate may be greater than or equal to about +3 mL/minute to less than or equal to about +7 mL/minute. For example, the inlet rate may be about +5 mL/minute. In at least one example embodiment, the circulation rate may be one-half of the total inlet rate. For example, when the inlet rate is about +5 mL/minute, the circulation rate may be about −2.5 mL/minute.

The inlet rate and the circulation rate may be held for a fifth time period. In at least one example embodiment, the fifth period may be greater than or equal to about 40 seconds (e.g., greater than or equal to about 50 seconds, greater than or equal to about 60 seconds, greater than or equal to about 70 seconds, greater than or equal to about 80 seconds, or greater than or equal to about 90 seconds). In at least one example embodiment, the fifth period may be less than or equal to about 100 seconds (e.g., less than or equal to about 90 seconds, less than or equal to about 80 seconds, less than or equal to about 70 seconds, less than or equal to about 60 seconds, or less than or equal to about 50 seconds). In at least one example embodiment, the fifth period may be greater than or equal to about 40 seconds to less than or equal to about 100 seconds. For example, the fifth period may be about 1.5 minutes (or 90 seconds).

The method 2000 may include causing 2020 the volume of fluid to move through at least a portion of the bioreactor-based system (and more specifically, the intracapillary space, and additionally, or alternatively, the extracapillary space). In at least one example embodiment, causing 2020 the volume of fluid to move through at least a portion of the bioreactor-based system may include introducing the volume of fluid to the bioreactor-based system. In at least one example embodiment, the volume of may be introduced to and flowed through the intracapillary space. In other example embodiments, the volume of fluid may be introduced to and flowed through the extracapillary space. In still other example embodiments, the volume of fluid may be introduced to and flowed through both the intracapillary space and the extracapillary space. In at least one example embodiment, causing 2020 the volume of fluid to move through at least a portion of the bioreactor-based system may include chasing the volume of fluid as it is introduced to the intracapillary space, the extracapillary space, or a combination of the intracapillary space and the extracapillary space.

The method 2000 may include preparing 2010 the volume of fluid including the cell population. In at least one example embodiment, preparing 2010 the volume of fluid including the cell population may occur before causing 2020 the volume of fluid to move through at least a portion of the bioreactor-based system (and more specifically, the intracapillary space, and additionally, or alternatively, the extracapillary space). In other example embodiments, preparing 2010 the volume of fluid including the cell population may concurrently with causing 2020 the volume of fluid to move through at least a portion of the bioreactor-based system (and more specifically, the intracapillary space, and additionally, or alternatively, the extracapillary space). In still other example embodiments, preparing 2010 the volume of fluid including the cell population may occur both before and during the causing 2020 of the volume of fluid to move through at least a portion of the bioreactor-based system (and more specifically, the intracapillary space, and additionally, or alternatively, the extracapillary space). In each instance, preparing 2010 the volume of fluid including the cell population may include loading a cell population with a fluid. For example, the cell population may be contacted to, or disposed in, the fluid. In at least one example embodiment, the volume of fluid may include a cell culture medium.

Although not illustrated, it should be appreciated that, in various example embodiments, the method 200 may include (or may occur after) the bioreactor-based systems (and more specifically, the intracapillary spaces, and additionally, or alternatively, the extracapillary spaces) are coated, for example, with recombinant laminin, recombinant vitronectin, or a combination thereof.

In various aspects, the present disclosure provides methods for expanding cells (like induced pluripotent stem cells, including those, loaded and distributed using, for example, the method illustrated in FIG. 5) using bioreactor-based systems (such as, the cell expansion system 10 illustrated in FIG. 1 and/or the bioreactor illustration illustrated in FIG. 2 and/or the bioreactor 501 illustrated in FIG. 4). FIG. 7 illustrates an example method 3000 for expanding cells (like induced pluripotent stem cells, including those, loaded and distributed using, for example, the method illustrated in FIG. 5) using bioreactor-based systems (such as, the cell expansion system 10 illustrated in FIG. 1 and/or the bioreactor illustration illustrated in FIG. 2 and/or the bioreactor 501 illustrated in FIG. 4).

The method 3000 includes an attaching phase 3010 where a cell population is bound to one or more surfaces of the bioreactor-based systems (and more specifically, the intracapillary spaces, and additionally, or alternatively, the extracapillary spaces).

In at least one example embodiment, the attaching phase 3010 may include causing bidirectional flow (which may also be referred to as counterflow) of fluid (e.g., cell culture medium) to (further) drive the cell population to the (optionally coated) the one or more surfaces of the bioreactor-based systems (and more specifically, the intracapillary spaces, and additionally, or alternatively, the extracapillary spaces). Bidirectional flow includes the movement of fluid (e.g., cell culture medium) from both the inlet (or first) end and the outlet (or second) end of the bioreactor-based systems. In at least one example embodiment, for example, when the volume of fluid including the cell population is flowed through the intracapillary space, bidirectional flow may include the movement of fluid (e.g., cell culture medium) from the outlet of the intracapillary space (e.g., the first outlet 28 as illustrated in FIG. 1 and/or the intracapillary outlet port 120 as illustrated in FIG. 2 and/or intracapillary outlet port 501B illustrated in FIG. 4) and also the inlet of the intracapillary space (e.g., the first inlet 22 as illustrated in FIG. 1 and/or the intracapillary inlet port 108 and/or intracapillary inlet port 501A illustrated in FIG. 4).

In at least one example embodiment, the attached phase 3010 may include causing or moving the fluid at ultralow flow rates from both the first and second ends (e.g., the intracapillary inlet and the intracapillary outlet). The ultralow flow rate of the fluid may allow opportunities for the binding of the cell population to the (optionally coated) one or more surfaces of the bioreactor-based systems (and more specifically, the intracapillary spaces, and additionally, or alternatively, the extracapillary spaces), without washing out the cell population.

During the attaching phase 3010, flow may be split between the first and second end. In at least one example embodiment, the flow may be evenly split between the first and second ends. In at least one example embodiment, the flow rates from each end during the attaching phase 3010 may be greater than or equal to about 0.02 mL/minute (e.g., greater than or equal to about 0.03 mL/minute, greater than or equal to about 0.04 mL/minute, greater than or equal to about 0.05 mL/minute, greater than or equal to about 0.06 mL/minute, greater than or equal to about 0.07 mL/minute, greater than or equal to about 0.08 mL/minute, or greater than or equal to about 0.09 mL/minute). In at least one example embodiment, the flow rates from each end during the attaching phase 3010 may be less than or equal to about 0.1 mL/minute (e.g., less than or equal to about 0.09 mL/minute, less than or equal to about 0.08 mL/minute, less than or equal to about 0.07 mL/minute, less than or equal to about 0.06 mL/minute, less than or equal to about 0.05 mL/minute, less than or equal to about 0.04 mL/minute, or less than or equal to about 0.03 mL/minute). In at least one example embodiment, the flow rates from each end during the attaching phase 3010 may be greater than or equal to about 0.02 mL/minute to less than or equal to about 0.1 mL/minute. The ultralow flow rate may continue for a sixth time period. The sixth period may be directly proportion to the flow rate.

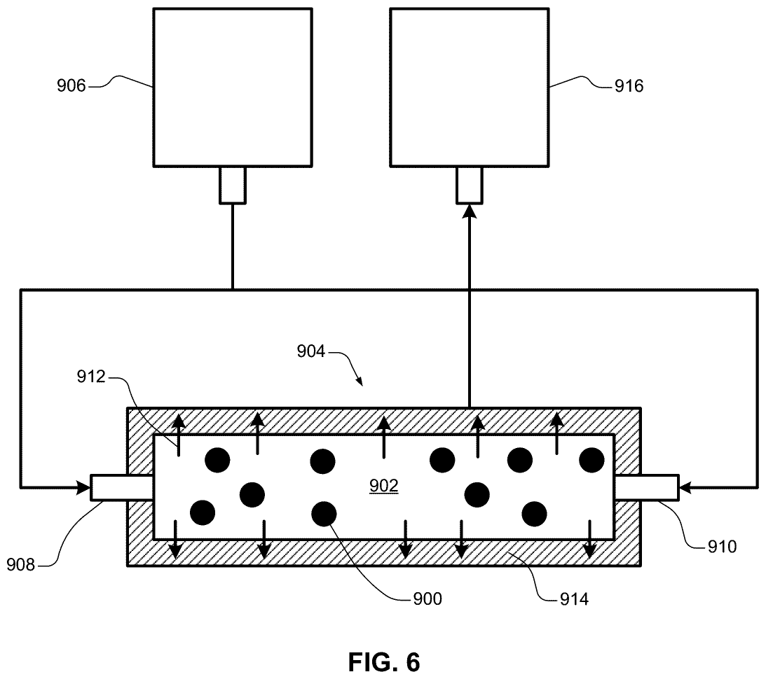

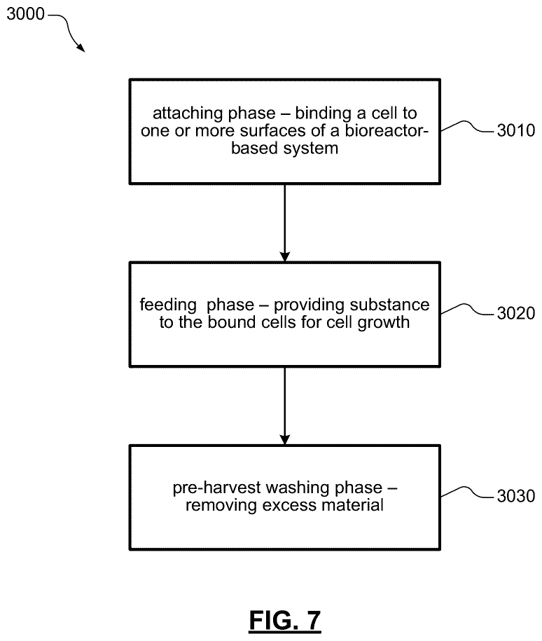

By way of example, FIG. 6 provides a schematic of an example counterflow or bidirectional containment scheme. As illustrated, cells and/or other materials 900 may be positioned (or repositioned) within an intracapillary portion or space 902 of a bioreactor 904. More specifically, as media or fluid may move from a media bag or container 906 it may be split between an intracapillary inlet pump (not shown) and an intracapillary circulation pump (not shown) such that the media or fluid moves into the intracapillary portion or space 902 of the bioreactor 904 through both an intracapillary inlet 908 and an intracapillary outlet 910. In contrast, the cells and/or other material 900 may be seeded and/or recirculated using unidirectional flow from the media bag or container 906 to the intracapillary portion or space 902 of the bioreactor 904 via the intracapillary inlet 908. Counter flows may be adjusted such that cells and/or other materials might be positioned or held at selected locations or area within the bioreactor 904 between inlet and outlet locations. Arrows 912 illustrate the fluid movement through pores of the bioreactor 904 from the intracapillary portion or space 902 to the extracapillary portion or space 914 during the counterflow containment. A waste bag 916 may be in fluid communication with the extracapillary portion or space 914 allowing appropriate movement from the extracapillary portion 914 to the waste bag 916. Although the cells and/or other materials 900 are illustrated in FIG. 6 as being seeded within the intracapillary portion or space 902, it should be appreciated that, in various other example embodiments, the cells and/or other materials 900 may instead be seeded instead in the extracapillary portion or space 914. In such instances counterflow containment may include moving media or fluid from the media bag or container 906 to an extracapillary inlet (not shown) and also an extracapillary outlet (not shown).

With renewed reference to FIG. 5, the method 3000 may include a feeding phase 3020 where substance for cell growth is provided to the cell population such as bound in the bioreactor-based systems (and more specifically, the intracapillary spaces, and additionally, or alternatively, the extracapillary spaces) during the attaching phase 3010. In at least one example embodiment, the feeding phase 2030 may occur after the attaching phase 3010. In other example embodiments, at least a portion of the feeding phase 2030 may overlap with, or occur concurrently with, at least a portion of the attaching phase 3010. That is, the sixth period of the attaching phase 3010 may overlap with a seventh period of the feeding phase 3020.

In at least one example embodiment, the feeding phase 3020 may include causing to move, or moving, the substance through the bioreactor-based systems (and more specifically, the intracapillary spaces, and additionally, or alternatively, the extracapillary spaces). In at least one example embodiment, the substance may be caused to be moved, or moved, through the bioreactor-based systems (and more specifically, the intracapillary spaces, and additionally, or alternatively, the extracapillary spaces) at a reduced rate that helps to limit the detachment of the cell population as bound in the bioreactor-based systems (and more specifically, the intracapillary spaces, and additionally, or alternatively, the extracapillary spaces) that may otherwise result from shear forces.

In at least one example embodiment, the flow rates during the feeding phase 3020 may be greater than or equal to about 0.5 mL/minute (e.g., greater than or equal to about 0.7 mL/minute, greater than or equal to about 0.9 mL/minute, greater than or equal to about 1.1 mL/minute, greater than or equal to about 1.3 mL/minute, greater than or equal to about 1.5 mL/minute, greater than or equal to about 1.7 mL/minute, or greater than or equal to about 1.9 mL/minute). In at least one example embodiment, the flow rates during the feeding phase 3020 may be less than or equal to about 2 mL/minute (e.g., less than or equal to about 1.9 mL/minute, less than or equal to about 1.7 mL/minute, less than or equal to about 1.5 mL/minute, less than or equal to about 1.3 mL/minute, less than or equal to about 1.1 mL/minute, less than or equal to about 0.9 mL/minute, or less than or equal to about 0.7 mL/minute). In at least one example embodiment, the flow rates during the feeding phase 3020 may be greater than or equal to about 0.5 mL/minute to less than or equal to about 2 mL/minute. For example, the flow rates during the feeding phase 3020 may be about 1 mL/minute. The flow may continue during the feeding phase 3020 for a seventh time period. The seventh period may be directly proportion to the flow rate.

The method 3000 may include a pre-harvest washing phase 3030 where excess fluid (including, for example, excess cell culture medium and/or feeding substance) may be removed from the bioreactor-based systems (and more specifically, the intracapillary spaces, and additionally, or alternatively, the extracapillary spaces). In at least one example embodiment, the pre-harvest washing phase 3030 may occur after the feeding phase 3020. In other example embodiments, at least a portion of the pre-harvest washing phase 3030 may overlap with, or occur concurrently with, at least a portion of the feeding phase 2030. That is, the seventh period of the feeding phase 3020 may overlap with an eighth period of the pre-harvest washing phase 3030.

In at least one example embodiment, the pre-harvest washing phase 3030 may include causing to move, or moving, a washing fluid through the bioreactor-based systems (and more specifically, the intracapillary spaces, and additionally, or alternatively, the extracapillary spaces). The washing fluid may include, for example, phosphate buffer saline (PBS). In at least one example embodiment, the washing fluid may be caused to be moved, or moved, through the bioreactor-based systems (and more specifically, the intracapillary spaces, and additionally, or alternatively, the extracapillary spaces) at a reduced rate that helps to limit loss of expanded cells while moving the excess materials to a waste line and/or waste bag.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A method for using a bioreactor-based system, the method comprising:
loading and distributing cells in a space of the bioreactor-based system, the loading and distributing of the cells in the space of the bioreactor-based system comprising:
circulating a volume of fluid including a cell population through the space of the bioreactor-based system in a first direction for a first period; and
after the first period, circulating the volume of fluid including the cell population through the space of the bioreactor-based system in a second direction that is different from the first direction for a second period.

2. The method of claim 1, wherein the method further comprises:
after the second period, circulating the volume of fluid including the cell population through the space of the bioreactor-based system in the first direction for a third period.

3. The method of claim 2, wherein the space of the bioreactor-based system has a first orientation during the circulating of the volume of fluid including the cell population through the space in the first direction for the first period.

4. The method of claim 3, wherein the space of the bioreactor-based system has a second orientation different from the first orientation during the circulating of the volume of the fluid including the cell population though the space in the first direction for the third period.

5. The method of claim 4, wherein the space of the bioreactor-based system has the first orientation during the circulating of the volume of fluid including the cell population through the space in the second direction of the second period.

6. The method of claim 5, wherein the method further includes, after the circulating of the volume of fluid including the cell population through the space of the bioreactor-based system in the second direction of the second period, and before the circulating of the volume of fluid including the cell population through the space of the bioreactor-based system in the first direction for the third period:
rotating the space of the bioreactor-based system from the first orientation to the second orientation.

7. The method of claim 2, wherein the method further includes, after the third period:
circulating the volume of fluid including the cell population through the space of the bioreactor-based system in the second direction for a fourth period.

8. The method of claim 7, wherein the space of the bioreactor-based system has a first orientation during the circulating of the volume of fluid including the cell population through the space in the first direction for the first period, and the first orientation during the circulating of the volume of fluid including the cell population through the space in the second direction for the second period.

9. The method of claim 8, wherein the space of the bioreactor-based system has a second orientation different from the first orientation during the circulating of the volume of the fluid including the cell population though the space in the first direction for the third period, and the second orientation during the circulating of the volume of fluid including the cell population through the space in the second direction of the fourth period.

10. The method of claim 7, wherein the volume of fluid including the cell population is circulated at a first rate in the first direction for the first period and the volume of fluid including the cell population is circulated at a second rate in the first direction of the third period, the second rate being the same as the first rate.

11. The method of claim 7, wherein the third period is the same length as the first period.

12. The method of claim 7, wherein the volume of fluid including the cell population is circulated at a third rate in the second direction for the second period and the volume of fluid including the cell population is circulated at a fourth rate in the second direction of the fourth period, the fourth rate being the same as the third rate.

13. The method of claim 12, wherein the fourth period is the same length as the second period.

14. The method of claim 1, wherein the method further includes:
expanding cells loaded and distributed in the space of the bioreactor-based system, wherein the expanding of the cells loaded and distributed in the space of the bioreactor-based system includes:
maintaining a cell culture medium in the space of the bioreactor-based system using bidirectional movement of the cell culture medium at a first rate from both a first end and an opposing second end of the space.

15. The method of claim 14, wherein the expanding of the cells loaded and distributed in the space of the bioreactor-based system further includes:
causing the cell culture medium to move through the space of the bioreactor-based system at a second rate that is less than the first rate.

16. The method of claim 15, wherein the second rate is greater than or equal to about 0.5 mL/minute to less than or equal to about 2 mL/minute.

17. The method of claim 14, wherein the expanding of the cells loaded and distributed in the space of the bioreactor-based system further includes:
causing a washing fluid to move through the space of the bioreactor-based system.

18. A method for using a bioreactor-based system, the method comprising:
circulating a volume of fluid including a cell population through a space of the bioreactor-based system in a first direction for a first period, the space having a first orientation for the first period;
after the first period, circulating the volume of fluid including the cell population through the space of the bioreactor-based system in a second direction that is different from the first direction for a second period, the space having the first orientation for the second period;
after the second period, circulating the volume of fluid including the cell population through the space of the bioreactor-based system in the first direction for a third period, the space having a second orientation different from the first orientation for the third period; and
after the third period, circulating the volume of fluid including the cell population through the space of the bioreactor-based system in the second direction for a fourth period, the space having the second orientation for the fourth period.

19. The method of claim 18, wherein the method further includes, after the second period and before the third period:
rotating the space from the first orientation to the second orientation.

20. The method of claim 18, wherein the method further includes:
maintaining a cell culture medium in the space using bidirectional movement of the cell culture medium at a first rate from both a first end and an opposing second end of the space of the bioreactor-based system;
causing the cell culture medium to move through the space of the bioreactor-based system at a second rate that is less than the first rate; and
causing a washing fluid to move through the space of the bioreactor-based system.
